(19) United States
(12) Patent Application Publication
(10) Pub. No.: US 2013/0018257 A1
(43) Pub. Date: Jan. 17, 2013

(54) Device For Administering Heat To A Human Or Animal Tissue, Vessel Or Cavity

(71) Applicant: Henri Mehier, Chamonix-Mont-Blanc (FR)

(72) Inventor: Henri Mehier, Chamonix-Mont-Blanc (FR)

(21) Appl. No.: 13/621,643

(22) Filed: Sep. 17, 2012

Related U.S. Application Data
(63) Continuation of application No. PCT/FR2011/050462, filed on Mar. 4, 2011.

(30) Foreign Application Priority Data
Mar. 23, 2010 (FR) 1052109

(51) Int. Cl. A61N 1/02; A61B 18/14; A61B 6/00; A61N 5/10

(52) U.S. Cl. 600/432; 607/99; 600/2

(57) ABSTRACT

Device for administering heat to a cavity, tissue or vessel, characterised in that it takes the form of an insulated copper wire over the length of which is crimped a tube having a resistivity of more than 20 μΩ·cm, the copper wire (1) is in contact with the said tube at its distal extremity, the tube is enclosed over part of its length by at least one movable cylinder having an electrical resistance of less than 20 μΩ·cm, and the movable cylinder and the copper wire have means for connection to a current generator.

### FIELD OF THE INVENTION

The invention relates to a new device for administering heat, or more specifically dry heat, into an accumulation of fluid, a cavity (bladder, etc.), tissue or vessel in humans or animals.

### BACKGROUND OF THE INVENTION

The device according to the invention may be used for the treatment of tumours by the technique known as thermonecrosis or hyperthermia.

In a known way, thermonecrosis involves the destruction of cells at a temperature above 65° C. and is more particularly used for localised tumours. This technique is employed using dry heat from a laser, RF or ultrasound. These different means may be used in acute intervention mode and the great amount of heat provided in a short time gives rise to a carbonisation phenomenon that may cause fatal shock, with the result that these techniques are only used for tumours of small size. With steam this carbonisation phenomenon is minimised, but the possible diffusion of condensation water at high temperature restricts application to tumours located in homogeneous tissues or large organs. None of these methods achieve complete necrosis and do not prevent recurrence. They are nevertheless unsuitable for localised tumours close to vital centres or organs (brain, mediastinum, oto-rhino-laryngeal area, etc.).

For its part hyperthermia is a technique that comprises increasing the temperature of the cells to a value of approximately 45° C., and does not result in their destruction but increases their ability for defence. This technique applies more particularly to tumours present in outflows of fluid. It is applied using short waves or liquids which have been heated by extracorporeal circulation.

There is therefore advantage in developing a single device that can be used for both technologies by programming the heat administration temperature, time and administration periods at the same time, as is the case in chemotherapy and radiotherapy, in order to optimise the desired effect, on an outpatient basis, without the patient being required to undergo further surgery and further hospitalisation. There is therefore an advantage in developing an outpatient method that can be used to progressively destroy tumours, even those of large size and located close to vital centres, in a programmed way, with the time to monitor the effect of the thermonecrosis or hyperthermia by imaging, and placing a sensor at the periphery of the tumour to measure the temperature of the adjacent healthy tissues.

The device according to the invention may also be used for the treatment of skin ageing, in particular for the treatment of wrinkles, through regeneration of the dermis by creating inflammation through a thermal effect resulting in scarring with regeneration of the collagen and myofibrils.

The device according to the invention may finally be used for the treatment of varicosities by creating fibrosis or thermofibrosis phenomena in the wall of the vein, resulting in its obliteration.

Hitherto the Applicant has developed a number of devices described in documents WO 00/29055, WO 03/070302, WO 2006/108974 that can inject heat through the administration of steam into tissues or vessels. The general principle common to all these devices is keeping the water in a pressurised form before its release, that is to say liquid at a temperature above 100° C., which makes it possible to have optimised heat transfer between the heat source and the water in pressurised form. In order to do this, in all the solutions proposed by the Applicant hitherto the water circulates within a restricted space, in particular within the bore of microtube of diameter between 100 and 250 μm.

More recently the Applicant has developed a different device described in document WO 2009/083688. This is a micro-resistance which heats the water present in the tumour or in the blood in varicose veins and therefore no external water is provided. The micro-resistance takes the form of a central constantan wire covered with a copper sheath, except at its distal extremity, and then with an insulating layer and a current-conducting tube over its entire length. Such a device makes it possible to generate heat only at the distal extremity of the micro-resistance. Apart from its high cost, the major disadvantages of the system are that the heating element is central and the heat must pass through the insulating element in order to diffuse within the body, with a loss of thermal efficiency and reliability. As the heating element is internal, the user cannot define the length heated or the position of the non-heating zones.

In all the systems described by the Applicant energy is provided by a standard non-independent generator located outside the body, preventing use of the device on an outpatient basis.

### SUMMARY OF THE INVENTION

The object of the invention is therefore to simplify this type of system to make a single low-cost device that is appropriate for all the types of treatment described above.

Another object of the invention is to provide a device that can be used on an outpatient basis, i.e. that is easily transportable and therefore independent.

Finally, none of the devices described previously is suitable for multiple-treatment use. In other words a third object of the invention is to develop a single system that is not only capable of delivering heat in the form of dry heat but can also dispense a standard therapeutic treatment in the form of a chemical or radioactive active ingredient.

In order to do this the Applicant has developed a new system, still in the form of a micro-resistance, in which the length of the heating zone may be determined in relation to and at the time of use.

More specifically the invention relates to a partly or wholly-implantable device intended to administer heat into a cavity, tissue or vessel in particular, characterised in that:
it takes the form of a copper wire that is insulated except at its distant and proximal ends, over at least part of the length of which there is a crimped a resistant tube having a resistivity of more than 20 μΩ·cm,
the copper wire is in contact with the said tube at its distal extremity whereas its proximal extremity is intended to be connected to a current generator,
the tube is enclosed over part of its length by at least one proximal movable cylinder that can be crimped onto the tube by the user, using for example pliers, the said cylinder having an electrical resistance of less than 20 μΩ·cm,
the movable proximal cylinder and the proximal extremity of the copper wire have means for connection to a current generator, in practice the proximal extremity of the copper wire is connected to the positive pole of the generator and the movable cylinder is connected to the negative pole of the generator through for example a second insulated copper wire.

In other words the invention comprises crimping a material returning the current and having high resistivity, which is manifest as a heating phenomenon at least partly onto an insulated wire carrying the current. A movable cylinder or ring of lower resistivity, which is not heating because it forms an electrical shunt for the resistant circuit is provided on this material, the position of the ring being determined at the time of use before being permanently crimped by the user using pliers. The cylinder will advantageously be positioned at the level of the skin and down to the zone that has to be heated, after the size of the tumour or vein, and therefore the zone that has to be heated, has been determined.

According to the invention the resistant tube having a resistivity of more than 20 μΩ·cm is crimped onto the copper wire by drawing or advantageously by micro-swaging during manufacture. In a preferred embodiment the wire is crimped by micro-swaging to provide the wire/tube assembly with a triangular profile.

The tube is at least partly crimped onto the wire. In an advantageous embodiment the tube is crimped onto the wire only at its distal extremity, preferably over a length of approximately 5 cm.

In an improved embodiment the device is provided with a second movable cylinder known as the “distal cylinder” which can be crimped in the same way at the time of use, the cylinder having a diffuser for an active ingredient at its porous surface.

In another embodiment the device has a second movable cylinder known as the “distal cylinder”, the surface of which is provided with a radioactive product for therapeutic and location purposes.

In both cases the cylinder as such may be made of a material of variable resistivity, which imparts a greater or lesser heating property to it according to the nature of the zone that has to be treated, and if necessary to activate diffusion of the active ingredient.

Of course the device according to the invention may be fitted with several cylinders whereby the diffusion of a chemical active ingredient or radiotherapy may be associated with the heat treatment. In all cases the more proximal cylinder is connected to the current generator.

In this case the operator has a system which is suitable for multiple therapy, and therefore capable of delivering dry heat and an active ingredient at the same time.

According to the invention the tube having a resistivity of more than 20 μΩ·cm is made of a material selected from a group comprising steel, stainless steel, nitinol or titanium.

As already mentioned, the tube is crimped by drawing or micro-swaging onto an insulated copper wire whose uninsulated distal extremity is in contact with the distal extremity of the said tube. Advantageously the copper wire is insulated by means of a layer of polyimide, Teflon®, or alumina.

In the remainder of the description of the term “distal” indicates the extremity of the tube in contact with the zone that has to be treated, whereas the “proximal” extremity indicates the part of the tube intended to be connected to the current generator.

According to another characteristic the material constituting the movable cylinder having an electrical resistivity of less than 20 μΩ·cm is selected from the group comprising brass or copper, which may be covered with a very thin layer of material of better biocompatibility such as gold deposited electrochemically by flash gilding.

Of course the tube must have a sufficiently small diameter to be incorporated in the body, in particular within vessels or tissues. In practice the diameter of the device lies between 0.3 and 0.7 mm, and is advantageously of the order of 0.5 mm, including the excess thickness created by the presence of the movable cylinder after crimping.

In a particular embodiment the tube takes the form of a spiral. This shape is particularly suitable for treating a larger volume without having to resort to the multiple emplacement of micro-resistances. In this case the diameter of the device is between 1 and 1.2 mm, to impart some rigidity to the spiral.

According to an essential characteristic, the tube is connected to a current generator. Depending upon the type of indication envisaged (varicosity, tumour, wrinkle), the tube may be heated continuously or in pulses.

The invention therefore also relates to a device associating a current generator with the device as just described.

In general the generator takes the form of an electrochemical generator of the “alkaline battery” or more highly developed type, which has the advantage of being self-regulated with regard to current and voltage, and therefore power, eliminating any danger to the body without having to resort to a complex electronic system. The battery is advantageously an alkaline battery. In an improved embodiment the functioning of the battery is programmed.

The electrochemical generator also has the advantage of having a weight/energy ratio very much superior to that of other independent systems for a very low operating cost per intervention. Depending upon the type of treatment envisaged and therefore the type of device according to the invention (also described as a “micro-resistance”) the basic voltage is adjusted by the number of batteries used in series.

In order to generate current pulses the electrical circuit is supplemented by a “supercapacitance” placed in parallel between the current generator and the micro-resistance. In this case the circuit includes a switch or a programmer, operating in inverter mode which charges the supercapacitance and then discharges it into the micro-resistance. With this system the energy delivered by each pulse is perfectly defined and always the same, without resort to a complex electronic system. The “supercapacitance”, generally of the DLC (double-layer electric field) type, which has a very high electrical capacitance, from several farads to several hundred farads, is well suited to the needs of this medical device and will charge and discharge a virtually unlimited number of times.

In any event the generator is advantageously independent.

The invention also relates to a method for the treatment of wrinkles by regeneration of the dermis using the device according to the invention.

More specifically, the method comprises:
determining the size of the wrinkle that has to be treated, and therefore the length of the heating part of the device according to the invention that has to be provided,
crimping the proximal movable cylinder of resistivity less than 20 μΩ·cm onto the proximal extremity of the heating zone so determined, over the remaining part of the tube,
introducing the heating part of the device beneath the skin, in the locality of the wrinkle, or positioning it on the surface of the said wrinkle,
connecting the device to the terminals of a circuit comprising a battery, a generator and a switch so as to generate pulses of current,
then triggering of the generator so that it delivers pulses of energy between 20 and 50 joules over a period of between one and five seconds,
withdrawing the device.

In practice the generator takes the form of 3 R20 batteries delivering a voltage of 4.5V with 18 AH, making it possible to deliver approximately 300 KJ, that is 6000 pulses. The supercapacitance system comprises two supercapacitances of 22 farads placed in series.

In practice the current pulses create microtraumatism giving rise to inflammation and a scarring phenomenon which regenerates the collagen and microfibrils.

In the same way as before, the device according to the invention may be used for the treatment of localised tumours by thermonecrosis or hyperthermia in the field of cancer.

In this case the length of the tube that has to be heated is determined in relation to the size of the tumour. The tube is then fitted with a proximal movable cylinder crimped at the moment of use as it passes through the skin. In a preferred embodiment the device is also provided with additional distal movable cylinders offering a diffuser support for the active ingredient so that multiple treatment can be provided.

The invention also relates to a process for the treatment of tumours through the administration of heat comprising:
defining the size of the tumour that has to be treated, and therefore the length of the heating part of the device according to the invention that has to be provided,
crimping the proximal movable cylinder of resistivity less than 20 μΩ·cm to the proximal extremity of the heating zone determined in this way, over the remaining part of the tube,
making an incision in the skin,
introducing the heating part of the device into the target tissue,
then triggering the generator so that it delivers energy pulses,
withdrawing the device at the end of treatment.

In some cases the device is introduced in a catheter that has been previously fitted into the tumour thus making it possible to make use of an “access” which is already in place.

In the same way as before the device according to the invention may be used for the treatment of varicosities. The principle is to heat the vessel directly by generating a sufficient amount of heat to increase the temperature of the water present in the vessel to a temperature of approximately 95° C.

In this embodiment the device is introduced into the vein within a heat-resistant catheter. The vein can therefore be drained of its blood, which can be replaced by water delivered by this same catheter, thus bringing about uniform diffusion of energy. Permanent monitoring by echodoppler makes it possible to specify the moment and the condition of obliteration of the vein in real time. At the end of the operation the device is withdrawn in one go.

More specifically the method comprises:
identifying the vein requiring treatment by marking the route of the vein and the opening for introduction of the device on the skin,
determining the size of the vein that has to be treated, and therefore the length of the heating part of the device according to the invention that has to be provided,
crimping the proximal movable cylinder of resistivity less than 20 μΩ·cm to the proximal extremity of the heating zone so determined, over the remaining part of the tube,
introducing the heating part of the device into the vein,
connecting the device to the terminals of the generator,
then triggering the generator so that it delivers energy continuously at between 100 and 150 joules per centimetre of the vein requiring treatment, thus bringing about obliteration of the vein.

### BRIEF DESCRIPTION OF THE DRAWINGS

The invention and the advantages deriving from it will be clearly apparent from the following embodiments supported by the appended figures.

FIG. 1 is a diagrammatical illustration of the equipment according to the invention in cross-section.

### DETAILED DESCRIPTION OF THE INVENTION

The device comprises a central copper wire (1) insulated by a layer of polyimide (2). The distal extremity (3) of the copper wire is bare and in contact with the distal extremity (4) of the tube (5) made of stainless steel. Tube (5) is itself covered with 3 movable rings (6, 7, 8). Each of these 3 rings is manufactured of brass. The rings are crimped at the time of use after the part of the tube that has to be heated, and therefore the remaining locations for the rings, has been determined. In practice ring (6) is positioned at the passage through the skin in order to prevent any risk of burning. Ring (7) is provided with a porous support for diffusion of an active ingredient, not shown. Finally ring (8) has a surface provided with a radioactive product.

The equipment according to the invention also includes an electrochemical current generator (9) and a supercapacitance (10).

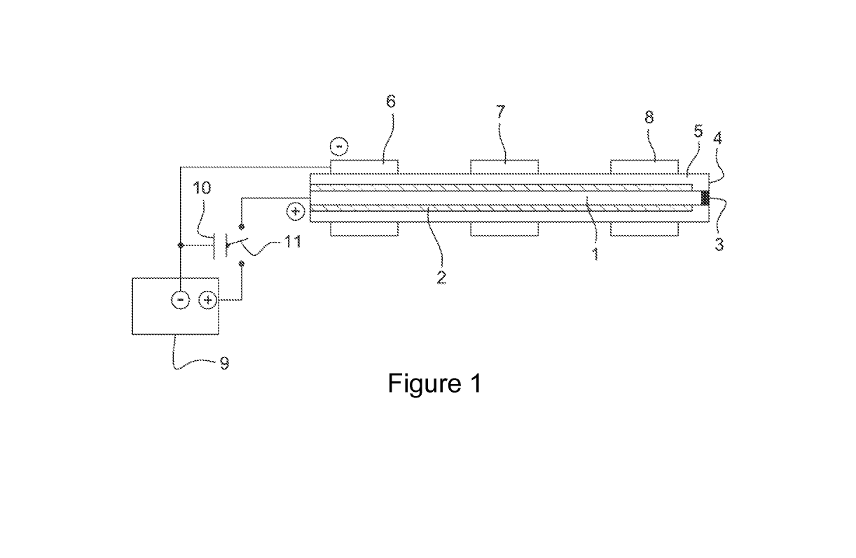

The flow of current is illustrated by the arrows in FIG. 1. The proximal extremity of copper wire 1 is connected to the positive pole of a generator through the copper wire whereas the negative pole of the generator is connected to the nearest proximal ring (5).

The generator takes the form of alkaline batteries. When it is used in pulsed mode the generator is associated with a supercapacitance (10) in parallel with the circuit, charging and discharging of the device being brought about through a switch (11) or a programmer operator in inverter mode.

As already mentioned, the device may be used for various treatments, whether for wrinkles, varicosities or in the field of cancer. Another advantage of the invention is that the device is provided with an independent battery which not only reduces its cost but also means that treatment can be provided on an outpatient basis.

What is claimed is:

1. Wholly or partly implantable device intended for the administration of heat into a cavity, tissue or vessel in particular, characterised in that:
it takes the form of a copper wire (1) that is insulated except at its distal and proximal extremities over at least part of the length of which there is crimped a resistant tube (5) having a resistivity of more than 20 μΩ·cm,
the copper wire (1) is in contact with the said tube through its distal extremity (3) while its proximal extremity is intended to be connected to a current generator (9),
the tube is enclosed over part of its length by at least one proximal movable cylinder (6) which can be crimped onto the tube (5) the said cylinder (6) having an electrical resistance of less than 20 μΩ·cm,
the movable proximal cylinder (6) and the proximal extremity of the copper wire have means for connection to a current generator (9).

2. Device according to claim 1, characterised in that the tube (5) is only crimped onto the wire at the distal extremity of the said wire, preferably over a length of approximately 5 cm.

3. Device according to claim 1, characterised in that the tube (5) is crimped by micro-swaging.

4. Device according to claim 1, characterised in that it is provided with a distal movable cylinder (7, 8) provided on its surface with a porous support which is a diffuser for an active ingredient.

5. Device according to claim 1, characterised in that it is provided with a distal movable cylinder (7, 8) provided on its surface with a radioactive product for therapeutic and location purposes.

6. Device according to claim 1, characterised in that the tube (5) which has a resistivity of more 20 μΩ·cm is made of a material selected from the group comprising steel, stainless steel, nitinol or titanium.

7. Device according to claim 1, characterised in that the copper wire (1) is insulated by means of a layer (2) of polyimide, Teflon®, or alumina.

8. Device according to claim 1, characterised in that the material comprising the proximal movable cylinder (6) having an electrical resistivity of less than 20 μΩ·cm is selected from the group comprising brass or copper.

9. Device according to claim 1, characterised in that it has a diameter of between 0.3 and 0.7 mm, advantageously of the order of 0.5 mm.

10. Equipment combining the device according to claim 1 with a current generator (9).

11. Equipment according to claim 10, characterised in that the current generator is an electrochemical generator.

12. Equipment according to claim 10, characterised in that it comprises a “supercapacitance” (10) placed in parallel between the current generator (9) and the device together with a switch (11) or programmer operating in inverter mode ensuring that the supercapacitance (10) charges up and then discharges in the device.
